(19) United States
(12) Patent Application Publication
(10) Pub. No.: US 2002/0058271 A1
(43) Pub. Date: May 16, 2002

(54) Process for detecting a nucleic acid target

(76) Inventor: Mark K. Johnson, New Berlin, WI (US)

Correspondence Address: Mark K. Johnson, PO Box 510644, New Berlin, WI, 53151-0644, US

(21) Appl. No.: 09/902,134

(22) Filed: Jul. 9, 2001

Related U.S. Application Data
(63) Continuation of application No. 09/612,150, filed on Jul. 7, 2000, now abandoned.

(51) Int. Cl. C12Q 1/68; C12P 19/34

(52) U.S. Cl. 435/6; 435/91.2

(57) ABSTRACT

Described is a process for detecting the presence or absence of one or more target nucleotides. A primer is added to a solution containing DNA. The solution is put under conditions favorable to PCR amplification using sequence specific primers. Background is removed and product is then measured to determine the presence or absence of target.

This application is a Continuation-In-Part of U.S. patent application, Ser. No. 09/612,150 which was filed on Jul. 7, 2000.

### FIELD

The present invention relates to a method and kit for detecting the presence or absence of a target nucleotide. The process is of particular interest in the testing of DNA samples for mutations, deletions and polymorphisms, or occurrences to the genome not inherited, such as environmentally induced mutations, deletions, substitutions and additions, and provides a general method for detecting point mutations. It is also useful in the detection and typing of infectious pathogens by analysis of their DNA.

### BACKGROUND

Several hundred genetic diseases are known to exist in man, which result from particular mutations at the DNA level. The molecular basis for certain of these diseases is already known and research is rapidly revealing the molecular basis for those genetic diseases for which the nature of the mutation is at present unknown. Where the precise molecular basis for the inherited condition is not known, diagnosis of the disorder or location of carriers may be provided in informative pedigrees by restriction fragment length polymorphism (RFLP) technology using DNA probes in genetic linkage with the disease locus.

Thus, at present Duchenne Muscular Dystrophy, Cystic Fibrosis and Huntington's Chorea among others may be diagnosed using RFLP technology. However, such testing needs to be performed separately in respect to each condition and a substantial amount of work is required, each case likely requiring DNA purification, restriction enzyme digestion, agarose gel electrophoresis, Southern blotting, hybridization, detection of hybridized gene probe and pedigree analysis. Certain other inherited conditions are known to be associated with single point mutations or polymorphisms in genes, but each of these conditions must be analyzed separately and further particular difficulties arise where the point mutations are heterogeneous. This can involve complex RFLP haplotype analysis with multiple restriction enzymes.

Polymorphisms in HLA sequences are also known to be associated with disease conditions. Proteins encoded by polymorphic loci are most commonly typed using serological methods. One of the limitations of serological typing is that it does not differentiate between many of the alleles that are known to exist in the population. This has prompted the development of methods for analysis of HLA as well as other allelic polymorphisms at the genetic level.

One of the oldest methods for typing, Southern Analysis, is based on immobilizing genomic DNA onto a solid phase, such as nitrocellulose or nylon membrane, and probing that material with a radiolabeled oligonucleotide “probe.” The nucleic acid sequence of the probe was selected to be complementary to a segment of the captured genomic material that included a known polymorphism. The ability to correctly interpret the test sample was dependent on the binding efficiency of the probe to the captured and denatured genomic DNA. In turn, the binding efficiency of the probe was dictated by the amount of time the probe was exposed to its potential target, as well as the composition and temperature of the hybridization buffer. In practice, in order to reduce non-specific binding of the probes, conditions are selected that slightly disfavor probe binding. Consequently, it is necessary to have a sufficient number of copies of target material and a very sensitive method of detection, as found with radiolabeled probes.

Coupling Southern analysis and restriction fragment length polymorphisms (RFLPs) made improvements to this strategy. The DNA was first digested by a restriction enzyme that cleaved at a specific sequence throughout the genomic material. The resulting fragments were then size-fractionated by gel electrophoresis prior to transfer to the membrane. The bound material was then visualized with a probe that would bind to a relevant genomic segment. So, if the formation or deletion of a specific restriction enzyme site could describe a polymorphism, then, following probing of the digested and transferred material, the resulting fragment pattern would provide the bulk of information for interpretation of the sample. With this strategy, the probes became a mechanism for visualizing the results and were not used to identify specific polymorphisms.

The role of the probe was eliminated by the introduction of the polymerase chain reaction (PCR), as described in U.S. Pat. No. 4,683,202, issued Jul. 28, 1987. With the PCR, analysis of test samples could be focused entirely on the segments of genomic DNA containing a mutation, deletion and/or polymorphism (target). Mimicking the process of DNA replication, oligonucleotides bind to complementary regions and “prime” DNA strand synthesis by a DNA polymerase. Cis-positioned primers limit the size of the segment that is produced. The reaction is repeated many times to generate large quantities of a particular segment of genomic DNA. The amplified material, subjected to the restriction enzyme, was in sufficient quantity to allow the resulting fragments to be visualized directly in a gel following electrophoresis and staining with dyes.

Unfortunately, the elimination or formation of a restriction site does not describe all targets. Consequently, for some targets, discrimination is again dependent on the performance of sequence-specific (allele-specific) probes. Nevertheless, because the PCR produced large quantities of a specific segment for analysis, the amount of time required for hybridization was reduced. Moreover, because both the oligonucleotide probe and target amplification product are of a defined size, could be produced in large quantities, and the availability of non-isotopic detection systems, several configurations for DNA testing developed.

One strategy reflects the original method of DNA analysis: the amplification product was bound directly to a solid phase support (membrane, microtiter well, etc.), denatured by heat or chemicals, and probed. This procedure may be recognized as the “dot-blot” method. Alternatively, one could create a “reverse dot-blot” strategy by first attaching the probe to the solid phase and subsequently introducing the denatured amplification product. More recently, researchers have used sequence-specific oligonucleotide (“SSO”) probe hybridization to perform HLA-Class II typing. That method entails amplifying a target region of an HLA locus using PCR, contacting the amplified DNA to a plurality of sequence-specific oligonucleotide probes under hybridizing conditions, and detecting hybrids formed with the amplified DNA and the sequence-specific oligonucleotide probes. An advantage of the reverse dot-blot method is that it enables multiple amplification products, produced in a single PCR, to be conveniently introduced to multiple separate probes previously aliquotted. A single test sample can be analyzed for multiple alleles, simultaneously. Unfortunately, this multi-allelic analysis presupposes that all probes will work under similar conditions of time and temperature.

An alternative strategy again eliminates the role of discriminating probes by incorporating specificity into the amplification reaction by using sequence-specific primers. This approach capitalizes on the lack of a 5′ editing function in Taq polymerase; the DNA polymerase most often used in the PCR. The absence of this enzymatic function enables a nucleotide mismatch at or near the 3′ end of a primer to prevent amplification of that primer, and the failure to form a PCR product. In this approach, primers are designed to terminate at or near the site of a known polymorphism with the ultimate base being distinctive for either the wild type or mutant base. The primers are said to be sequence-specific (SSP) or allele-specific (ASP). Following PCR, the presence or absence of an amplification product indicates the presence or absence of an allele in the genomic sample. However, it should be appreciated that the disruptive nature of a base mismatch does not ensure that an amplification product will not be formed. Consequently, subjective interpretation of signal intensity may be required when analyzing results and assigning genotype to a test sample.

PCR products are, traditionally, size-fractionated by gel electrophoresis and visualized in the gel. As described, the gel endpoint is amenable to identifying multiple PCR products formed in a single reaction. In practice, constraining primer position and sequence to obtain a product of a specific size can negatively impact the yield of product, fragment visualization, and, subsequently, genotype assignment.

The present invention addresses some of the shortcomings of known methods and provides a new process for detecting DNA targets.

### SUMMARY

By selecting the nucleotide sequence of an oligonucleotide primer appropriately it is possible to selectively achieve primer extension of a sequence containing a target or to prevent or subdue such primer extension.

Provided is a method for detecting the presence or absence of at least one target nucleotide in one or more nucleic acids contained in a sample by treating the sample with appropriate nucleoside triphosphates, a compound for polymerization of the nucleoside triphosphates and a detection primer for a target sequence, the nucleotide sequence of the detection primer being such that it is substantially complementary to the target, whereby an extension product of the detection primer is synthesized when the detection primer is complementary to the corresponding nucleotide in the target sequence and no or less extension product is synthesized when the detection primer is not complementary to the corresponding target sequence; and determining the presence or absence of the target from detecting the extension product.

While the method of the present invention is of particular interest in detecting the presence or absence of at least one specific nucleotide (e.g. mutations, polymorphisms, deletions etc.) in a preferred embodiment. For example, using the method described, one may choose to detect a DNA sequence that is different by at least one base from a known wild type sequence. The difference could be a deletion of one or more nucleotide bases, substituted bases, or even bases added to the genomic sequence to be detected. The difference may be attributable to an inherited mutation, deletion, substitution, addition, or polymorphism or it may be attributable to incidents to the genome other than genetic inheritance, such as a change of one or more bases to a known genomic sequence or foreign DNA incorporated into a cell.

A kit for testing DNA for at least one target nucleotide, whether inherited or not inherited, comprising: a receptacle containing a primer having a nucleotide sequence substantially complementary to a sequence of the DNA and a receptacle containing a reporter.

### DETAILED DESCRIPTION

The term “nucleoside triphosphate” is used to refer to nucleosides present in either DNA or RNA and thus includes nucleosides which incorporate adenine (A), cytosine (C), guanine (G), thymine (T) and uracil (U) as base, the sugar moiety being deoxyribose or ribose. In general deoxyribonucleosides will be employed in combination with a DNA polymerase. However, other modified bases capable of base pairing with one of the conventional bases adenine, cytosine, guanine, thymine and uracil may be employed. If desired one or more of the nucleoside triphosphates present in the reaction mixture for the purpose of incorporation in to the extended primer(s) may be labeled or marked in any convenient manner.

The term “nucleotide” as used can refer to nucleotides present in either DNA or RNA and thus includes nucleotides which incorporate adenine, cytosine, guanine, thymine and uracil as base, the sugar moiety being deoxyribose or ribose. It will be appreciated however that other modified bases capable of base pairing with one of the conventional bases, adenine, cytosine, guanine, thymine and uracil, may be used in the detection primer employed in the present invention.

The enzyme for polymerization of the nucleoside triphosphates may be any compound or system which will function to accomplish the synthesis of primer extension products, including enzymes. Suitable enzymes for this purpose include, for example, E. coli DNA Polymerase I, Klenow fragment of E. coli DNA polymerase I, T4 DNA polymerase, other available DNA polymerases, reverse transcriptase, and other enzymes, including thermostable enzymes such as Taq polymerase. The term “thermostable enzyme” refers to an enzyme which is stable to heat and is heat resistant and catalyzes (facilitates) combination of the nucleotides in the proper manner to form the primer extension products which are complementary to each nucleic acid strand. Generally, the synthesis will be initiated at the 5′ end of each primer and will proceed in the 3′ direction along the template strand, until synthesis terminates, producing molecules of different lengths. There may be thermostable enzymes for example which initiate synthesis at the 3′ end and proceed in the other direction, using the same process as described above.

The expression “target” means that portion of a DNA sequence which contains at least one nucleotide of interest, whether normal, a deletion, addition, substitution, polymorphism or other; the presence or absence of which is being detected by the described process. Generally one of possibly a plurality of potential target nucleotides will be a pairing base on the genomic strand opposite the 3′-terminal end of the primer extension sequence since, in a preferred embodiment, primer extension products will be initiated at the 5′ end of each primer as described above. The 3′-terminal end may include one or more 3′ bases in the primer. Where, however, an enzyme for polymerization is to be used which initiates synthesis at the 3′ end of the detection primer and proceeds in the 5′ direction along the template strand until synthesis terminates the appropriate sequence will contain the target near or at its 5′ end.

The term “oligonucleotide” as used herein is defined as a molecule comprised of two or more deoxyribonucleotides or ribonucleotides, preferably more than three. Its exact size will depend on many factors and the exact sequence of the oligonucleotide may also depend on a number of factors as described.

The term “primer” as used herein refers to an oligonucleotide, whether occurring naturally or produced synthetically, which is capable of acting as a point of initiation of synthesis when placed under conditions in which synthesis of a primer extension product which is complementary to a nucleic acid strand is induced, i.e., in the presence of appropriate nucleoside triphosphates and an enzyme for polymerization such as DNA polymerase. An appropriate buffer (“buffer” includes pH, ionic strength, cofactors, etc.) may be used at a suitable temperature.

The primer is preferably single stranded for maximum efficiency in extension, but alternatively may be double stranded. If double stranded, the primer is first treated to separate its strands before being used to prepare extension products. Preferably, the primer is an oligodeoxyribonucleotide. The primer must be sufficiently long to prime the synthesis of extension products in the presence of the enzyme for polymerization. The exact lengths of the primers will depend on many factors, including temperature and source of primer and use of the method. For example, depending on the complexity of the target sequence, the detection primers typically contain 12-40 nucleotides, although they may contain more or fewer nucleotides. Short primer molecules generally require lower temperatures to form sufficiently stable hybrid complexes with the template.

The term “complementary to” is used herein in relation to nucleotides to mean a nucleotide which will base pair with another specific nucleotide. Thus adenosine triphosphate is complementary to uridine triphosphate or thymidine triphosphate and guanosine triphosphate is complementary to cytidine triphosphate. It is appreciated that while thymidine triphosphate and guanosine triphosphate may base pair under certain circumstances they are not regarded as complementary for the purposes of this specification. It will also be appreciated that while cytosine triphosphate and adenosine triphosphate may base pair under certain circumstances they are not regarded as complementary for the purposes of this specification. The same applies to cytosine triphosphate and uracil triphosphate.

The primers herein are selected to be substantially complementary to the different strands of each specific sequence to be used as a template. This means that the primers must be sufficiently complementary to hybridize with their respective strands. Therefore, the primer sequence need not reflect the exact sequence of the template. For example, where the primer comprises a nucleotide sequence complementary to the target a non-complementary nucleotide fragment may be attached to the 5′-end of the primer, with the remainder of the primer sequence being complementary to the target sequence. The primers may utilize non-complementary nucleotides at a predetermined primer 3′ end to regulate efficiency of extension.

In certain circumstances synthesis of a detection primer extension product might be induced to occur even in the presence of a non-complementary 3′-terminal base or non-complementary base near the 3′ end. This result may arise from the use of too low a temperature in which case the temperature may be increased, too long a time of incubation/annealing in which case the time may be reduced, too high a salt concentration in which case the salt concentration may be reduced, too high an enzyme concentration, too high a nucleoside triphosphate concentration, an incorrect pH or an incorrect length of oligonucleotide primer or simply enzyme error. A major source of incorrect extension products may be allowing the reaction temperature to fall too low, thus permitting too low a stringency.

In addition to the above it may be found that incorrect results may also arise from use of a detection primer which is particularly rich in G (guanosine) and C(cytidine) residues. A detection primer may give rise to difficulty in this regard if it is G/C rich as a whole or particularly if it is G/C rich at its relevant, normally 3′, end. Moreover the precise nature of the base pairing in the region of the relevant, normally 3′, end of the detection primer when in use may be the cause of an incorrect result. Therefore, the presence of A's (adenosine) in the base pairing in the region of the relevant, normally 3′, end of the detection primer tends to improve specificity while the presence of G's (guanosine) does not. Furthermore the precise nature of the mismatch at the relevant, normally 3′, end of the detection primer may be an important factor in whether or not an inaccurate result is obtained. Thus for example an AA or CT mismatch does not normally result in hybridization, but a GT or AC mismatch may result in a sufficient degree of hybridization to result in the formation of inappropriate product(s). Inaccurate results may be avoided by deliberately introducing one or more mismatched residues, or if desired, deletions or insertions, within the detection primer to further reduce binding during hybridization.

In a preferred embodiment, a sample of DNA possibly containing a target sequence is tested. The DNA may be double or single stranded. Typically the DNA will be obtained from an organism. Preferably membrane material is disrupted to allow primers access to the DNA. Additionally, the removal of RNA may assist detection. A primer specific for a target sequence is added to a solution containing the DNA along with an extension polymerase and dNTP's for extension. No label is required at this point, however, dNTP's may be labeled by using radioactive isotopes to label DNA fragments. Alternatively, dNTP's associated with fluorescent dyes may be used as labels that fluoresce when excited, usually when exposed to light of a certain wavelength. The DNA is then put into a condition which is amenable to extension of the primer by the polymerase (e.g. denaturing double-stranded DNA, changing heat conditions, adjusting pH etc.). The primer is efficiently extended (by appropriate means including adding a polymerase and adjusting the temperature) when the target is present. The primer is designed to efficiently extend along the DNA template potentially containing target when the target is present. However, the primer is designed to prevent extension or allow inefficient extension when the target is not present. Detection of amounts of efficient primer extension product compared with detection of amounts of inefficient primer extension product determines if target is present. Efficient primer extension will yield a greater amount of double-stranded product than inefficient primer extension. Means of detection include measuring the emissions from labels associated with extension product.

In a preferred embodiment, a sample of cells is collected from a subject to be tested, such as by a buccal scrape containing cells or a blood sample. Any appropriate collection tool may be utilized which will collect a sufficient number of cells. Any known means may be used to allow the primer to associate with the genomic DNA such as disrupting the cell membrane or lysing the cell.

A target specific primer is added to a solution containing the genomic DNA that will substantially hybridize to a target located on the DNA. The primer is designed to either extend along the DNA template if a specific sequence is present or not extend if the specific sequence is absent. Since various conditions dictate whether or not the primer will extend, the primer may be designed to be substantially complementary or completely complementary and yet be expected to extend efficiently under either condition.

A polymerase is added to the solution containing the primer and the scraped material. Conditions are made to allow the primer to extend along the DNA template containing the target sequence if it is present. Alternatively, if the target sequence is not present the primer does not extend as efficiently. It is known that the primer may sometimes extend even in the absence of a target sequence, however, this extension is designed to be less efficient than that of the target sequence.

In a preferred embodiment, extension of the primer is detectable by adding a label that provides a signal when it encounters double-stranded DNA. Examples of such a labels are ethidium bromide, acridene orange, Sybr Green and Pico Green from Molecular Probes, Inc., Eugene, Oreg. The signal is measured by an appropriate means and target is detected. Pico Green is an example of a fluorescence emitting label. Pico Green intercalates with double-stranded DNA and emits a fluorescence light at a defined wavelength. When the label is excited, the emitted light is detectable as indicative of double stranded DNA. Additionally, a label that indicates whether single or double stranded DNA is present may be used since in a preferred embodiment, the amount of DNA is detected.

In another preferred embodiment, extension of the primer is detectable by using fluorescently labeled dNTP's. If efficient extension of the primer occurs, the labeled dNTP's will be incorporated into double-stranded DNA. The unincorporated dNTP's may be removed by means known to the art such as a spin column designed to collect small particles (such as dNTP's) yet allowing larger particles such as DNA to pass through. The labeled dNTP's incorporated into the extension product may then be caused to fluoresce and the light measured by appropriate means such as a fluorometer.

As stated earlier, even if target is not present, the primer may extend inefficiently. However, a comparison can be made between the signal detected from an extension product of a target sequence and the signal detected from an extension product when the target sequence is not present to determine if efficient extension occurred. Efficient extension is determined when the detected signal from the target extension product is measured at an amount statistically different from the detected signal from an extension product not containing the target sequence.

The same process can be applied to any sample of DNA whether purified or not. The source of the DNA does not appear to be important for the processes described. An important part of this specification is the ability to detect a target that may be as small as one nucleotide. A primer's efficient extension compared to inefficient extension is all that is required and usable results may be obtained from non-purified starting material including DNA contained in whole blood, lymphocytes, other cells, viruses, and bacteria.

In another preferred embodiment, genomic DNA is obtained from whole blood. The DNA may be purified using a purification method known to the art such as the Qiagen system. However, other purification systems are sufficient as well as no purification of the cellular DNA. The genomic DNA obtained is heated for a time sufficient to denature the double-stranded DNA. Then a primer which is specific for a target sequence located in the DNA is added. An extension polymerase is added along with dNTP's and the mixture is changed to a temperature that allows the primer to anneal to the DNA template and extend if the target sequence is present at a rate more efficient than if the target sequence were not present. The mixture is cooled and a label is added that provides a signal when double-stranded DNA is present.

It is believed that when any DNA to be tested using the described process requires denaturing, there will not be complete denaturing and some double-stranded portions of DNA not related to a target sequence will be present. Additionally, a primer is likely to be inefficiently extended even when the target sequence is not present resulting in double-stranded DNA. However, neither of these situations prevents target detection when the process is performed as described.

In another preferred embodiment, the target base may be a gene polymorphism. In this instance the target base is one of two choices. For example, for a specific polymorphism, perhaps 90% of the human population has the target base A but 10% of the population has the target base C. To detect the polymorphism, a primer is developed that is substantially complementary to the DNA sequence containing the polymorphism wherein the terminal base of the primer is a G which is in position to either anneal to the target base or not anneal if the target base is not present. If the sample of DNA tested contains the more prevalent target base A, the G will not anneal. If it contains the target base C, the complementary G will anneal. A polymerase is used to perform a polymerase chain reaction (PCR) to extend the primers in solution with the DNA to be tested. If the target base is A, the G does not anneal and the partially annealed primer will not be efficiently extended and will not produce as much extension product as the complementary T. If the target base is C, the G will anneal and the primer will be fully annealed and it will be extended efficiently when compared to a terminal base of T. Therefore, in this example, the presence of extension product indicates the presence of target base in the sample DNA. One can manipulate the terminal bases on the primer to obtain an extension product detecting the presence of any potential target base.

Detection methods previously described may be used to detect the presence of extension product in the prior examples. A dye (such as Molecular Probes PicoGreen or OliGreen) is added to the solution as previously described. The solution is then placed into a detector that can excite the dye to produce a detectable wavelength emission. Examples of detectors include but are not limited to flowcytometers (Beckton-Dickenson) and the Luminex 100 (Luminex Corporation). Detection of emissions indicates that an extension product is present.

In another preferred embodiment, the particle itself contains a detectable label, preferably a fluorescent signal emission. Therefore, the particle will emit a signal when appropriately excited as well as any attached fluorescently dyed extension product. In this manner, many different primers complementary to their unique portions of the sample DNA are used to produce multiple extension products, each of which will hybridize to specific oligonucleotides attached to particles. Each particle is manufactured to contain a unique signal that is specific to the individual sequence of the oligonucleotide attached. The detector can detect the unique particle signal in addition to the signal provided by the dyed extension product. Therefore, the particle is identified with extension product. If no extension product is present, a particle emission will be detectable but no extension product emission will be produced and the detector provides data indicating no extension product associated with a particular particle.

In another preferred embodiment, beads or other particles contain attached primers for producing extension product. The particles are added to a mixture of sample DNA prepared for the purpose of allowing extension product. The primers, which are unique for a particular target containing (or adjacent) sequence, anneal to their complementary sequence to either produce or not produce extension product according to previously described methods. After extension according to previously described methods, the mixture is heated to a temperature that allows the extension product and primer to disassociate from the template DNA. Particles are then detectable as well as any extension product attached. Again, multiple primers may be used as described.

In another preferred embodiment, sample DNA template containing target is placed in a solution prepared for extension product. Extension product is produced according to any of the previously described processes. After extension conditions are employed, the solution is heated to a temperature sufficient to allow disassociation of extension product, if any, from template. The solution is then added to separation process that separates genomic DNA from extension product and also removes a large percentage of remaining primers not extended (Wizard Preps, Promega Corporation). The separation process produces a solution of higher concentration extension product, if any extension product is made, relative to the solution before separation. The extension product is then dyed with a fluorescence emitting label and is detectable by known methods including flowcytometry and cytofluor detection.

In another preferred embodiment, PCR product may be detected by absorption. In this case, an added label is not required.

In another preferred embodiment, the presence or absence of at least one target nucleotide in a DNA sequence is determined. A first primer is added to a solution containing a first sample of genomic DNA. A second primer is added to a solution containing a second sample of the same genomic DNA. The second primer is substantially similar to the first primer except for one or more of the last 3 bases at the 3′ end. In most cases the last base differs between the primers. A polymerase, such as Taq polymerase, is added to each sample.

In some instances a single base polymorphism is the target base. Therefore, the first primer will contain one of the two potential bases at its 3′ end. The anti-sense complementary base to the polymorphism may also be utilized as the target base. The primers are configured to efficiently extend if the 3′ end anneals to the sample DNA and not extend or inefficiently extend if the 3′ end does not anneal. The primers may be manufactured to contain not only mismatches at the 3′ end but mismatches at the penultimate base or a prior base depending upon the sequence that allows efficient extension if the target base is present and inefficient extension if the target base is not present. Typically, only the final three bases at the primer 3′ end will be manipulated and the rest of the primer will be complementary.

After amplification, a significant amount of background will be present in each sample. The background is due to partial extension of primers at the target site or to primers non-specifically extending at other sites on the sample DNA or to RNA present or various other reasons. The background is so intense that it will completely disrupt any useful detection using the present method. Therefore, a background reducer must be utilized.

A background reducer is added to each sample after amplification. The background reducer may consist of a nucleic acid cleaving agent such as a nuclease. The preferred cleaving agent in the following examples is an endonuclease that prefers to cleave single stranded DNA or RNA. Two well known endonucleases used for that purpose are S1 nuclease and Mung Bean nuclease. It is believed that the non-specific product created during amplification is mostly single stranded nucleic acids which are a good mark for the single stranded preferring endonucleases. The single stranded nucleic acid bonds are cleaved to yield individual nucleotides leaving the double stranded amplification product intact.

After nucleic acid digestion, the remaining double stranded DNA is detected either by spectrophotometer absorption or by light emitting labels. A preferred label is a fluorescence emitting dye that emits fluorescence more intensely when in contact with nucleic acids as opposed to less intensely when in contact with nucleotides. Many dyes are available that react with nucleic acids, however, the preferred dyes are Pico Green and Oligreen (Molecular Probes), acridine orange and ethidium bromide. The dye is added immediately after nuclease digestion or the digestion product may be stored for detection later. The dye will fluoresce with more intensity if amplification product is abundant and with less intensity if amplification product in lesser amounts is present.

In this manner, one sample will cause the dye to produce more light than the other sample if one polymorphism nucleotide is present such as in homozygous genomic DNA samples. However, if a heterozygous DNA sample is present, the amount of light produced by the dye should be similar in each sample or at least more than a baseline standard amount of predetermined light intensity. Similarly, this process could be used with a single sample with a baseline standard light intensity reading. In other words, if the amount of light intensity detected from the dye is greater than the baseline intensity, the target is present. If the intensity is less than baseline, the target is not present. In this manner, deletions, mutations, genetic patterns etc. can be detected.

In the following examples, S1 endonuclease in a standard buffer (Promega, Madison, Wis.) is added to the PCR product to digest single-stranded DNA and RNA.

Examples of the present invention are provided for illustrative purposes and not to limit the scope of the invention.

### EXAMPLE 1

### Sample Preparation

Draw several mLs of venous blood into an EDTA tube and mix thoroughly by gently inverting the tube several times. Blood may be stored at 2-8° C. for several days before processing. Extract the DNA using either published techniques or a commercially available kit. Note that the method used for DNA isolation may dictate the volume and storage conditions of the blood. Resuspend the DNA in distilled water or 10 mM Tris (pH 7.0-7.3) to a concentration of 50 to 500 ng/μL. DNA that cannot be used immediately may be held at 2-8° C. for several days. For longer-term storage, samples should be stored frozen in a constant-temperature freezer. Excess contaminating protein, heparin, or EDTA may interfere with PCR extension of the purified DNA.

### EXAMPLE 2

### Compositions

Master Extension Mix contains next three (3) lines of materials

10 mM Tris (pH 8.3), 50 mM KCl, 0.01% Gelatin: Sigma [St Louis, Mo.]

200 μM dNTP(each): Pharmacia Biotech [Piscataway, N.J.]

0.5 μM primers: Custom synthesis from Genosys [The Woodlands, Tex.]

Primer 1 sequence=5′ 15 bases complementary to human HPA1 section+TC T 3′

Primer 2 sequence=5′ 15 bases complementary to human HPA1 section+TC C 3′

The 3′ T in primer 1 is complementary to the opposing base in HPA1. However, the 3′ C is not complementary if the A is present, therefore, promoting no extension or inefficient extension. Boil 20-500 ng/μL genomic DNA for 1 minute to denature. 0.5 units/μL Taq polymerase: Perkin Elmer [Foster City, Calif.], Promega (Madison, Wis.)

Prepare 90 μL of a fresh working dilution of Taq DNA polymerase (final concentration of 0.2 U/μL) with molecular biology grade water in a microfuge tube placed on ice.

| | | |
|---|---|---|
| Number of samples: | 4 | |
| Number of controls | 4 | |
| Add two for pipetting errors | 2 | |
| Total Number of Reactions | 10 | |
| Number of reactions | 10 | |
| Amount of Taq per reaction | × .5 | units |
| Total Units of Taq Required | 5.0 | units |
| Total units of Taq required | 5.0 | |
| Concentration of Taq stock | ÷ 5 | U/μL |
| Total Volume of Taq stock Required | 1 | μL. |
| Total volume required | 90 | μL |
| Volume of Taq | − 1.0 | μL |
| Volume of Water Required | 89.0 | μL |
| Total number of reactions | 10 | |
| Volume of Extension Mix per reaction | × 25 | μL |
| Total Volume of Extension Mix to Add to the Diluted Taq | 250 | μL |

Add 250 μL of refrigerated Extension Mix to the microfuge tube containing the 90 μLs of freshly diluted Taq DNA polymerase and vortex briefly to mix. Final volume equals 340 μL. If not extending 8 samples, then adjust the appropriate number in the previous equation to determine the required volume.

Pipette into each extension tube (held on ice):

35 μL Taq and Extension Mix solution: 2 tubes with primer 1; 2 tubes with primer 2; 2 tubes with no primer; 2 tubes with no Taq and primer 1;

1-15 μL boiled Genomic DNA (deliver ˜50 ng of isolated DNA per tube) 0-14 μL Water to bring final reaction volume to 50 μL

Cap tubes and temporarily store on ice.

Perform extension using the following protocol. Transfer the chilled extension tubes to a heating device: in the present example a thermal cycler was utilized.

| Cycle | Denature | Anneal | Extend |
|---|---|---|---|
| 1 | 94° C.-1 minute | | |
| 2-34 | 94° C.-5 seconds | 60° C.-15 seconds | 72° C.-15 seconds |

| After cycle 34 | Temperature | Time |
|---|---|---|
| Add S1 endonuclease in buffer | 65° C. | For 5 minutes |
| supplied by Promega (Madison, WI) | | |
| Then detect or freeze for later detection | | |

### EXAMPLE 3

### Detection

The extension products (˜25 μL each) are put into wells of a microtiter plate. 3 wells in each column contain primer 1 extension products; 3 wells in each column contain primer 2 extension products; 6 control wells contain a false primer; 6 control wells contain no Taq and primer 1. 150 μL of PicoGreen dsDNA Quantitation Reagent (Molecular Probes, Eugene, Oreg.) is added to each well after diluting according to instructions provided. PicoGreen intercalates with double-stranded DNA and then emits a fluorescent light. The plate is placed in a fluorometer capable of reading microtiter plates: excite at 480 nm and read at 520 nm. Readings can be taken immediately.

PCR Product using ˜50 ng genomic DNA per well

| known | | | | | |
|---|---|---|---|---|---|
| blood | HPA | HPA | HPA | | − Taq + |
| sample | a/a | a/b | b/b | − Primer | Primer 1 |
| Primer #1 | 90634 | 50881 | 12553 | 11354 | 10782 |
| Primer #1 | 94552 | 54650 | 12142 | 10683 | 11113 |
| Primer #1 | 91876 | 53852 | 11560 | 11231 | 12431 |
| Primer #2 | 18973 | 87345 | 98023 | 10892 | 11466 |
| Primer #2 | 21998 | 91384 | 99447 | 11345 | 10855 |
| Primer #2 | 17462 | 89311 | 97284 | 10978 | 11132 |

Numerous assays have been performed using different blood samples containing native genomic DNA having a known sequence. The same primers were used each time: one containing a 3′ terminal match to the HPA wild type and one containing a 3′ terminal match to the HPA polymorphism. The tests yielded similar definitive results in each case.

The foregoing is considered as illustrative only of the principles of the invention. Furthermore, since numerous modifications and changes will readily occur to those skilled in the art, it is not desired to limit the invention to the exact construction and operation shown and described. Accordingly, all suitable modifications and equivalents fall within the scope of the invention.

I claim:

1. A process for detecting the presence or absence of at least one target nucleotide in a DNA sequence, comprising:
a) adding a first primer to a solution containing a first sample of the DNA;
b) adding a second primer, which differs from the first primer by at least one base within 3 bases from and including a 3′ terminal base, to a solution containing a second sample of the DNA;
c) adding a polymerase to each sample;
d) performing PCR amplification;
e) reducing background nucleic acids using a background reducer;
f) measuring an amount of DNA from each sample; and,
g) comparing the measurements of each sample for determining if the target is present.

2. A process for detecting PCR amplification product, comprising:
a) performing a PCR amplification reaction;
b) adding a background reducer to the PCR amplification product;
c) detecting an amount of DNA present; and,
d) comparing the amount of DNA detected to a baseline standard to determine if amplification product is present.

3. A kit for using the process of claim 1 or 2, comprising: a receptacle containing a background reducer.
